(19) United States
(12) Patent Application Publication
(10) Pub. No.: US 2017/0135623 A1
(43) Pub. Date: May 18, 2017

(54) MUSCLE DETECTOR, MASSAGE APPARATUS AND METHOD FOR DETECTING MUSCLE

(71) Applicants: BOE TECHNOLOGY GROUP CO., LTD., Beijing (CN); BEIJING BOE OPTOELECTRONICS TECHNOLOGY CO., LTD., Beijing (CN)

(72) Inventors: Rui XU, Beijing (CN); Xue DONG, Beijing (CN); Hailin XUE, Beijing (CN); Xiaochuan CHEN, Beijing (CN); Xi CHEN, Beijing (CN); Jianyun XIE, Beijing (CN); Jian WANG, Beijing (CN); Lei MA, Beijing (CN); Xue CAO, Beijing (CN); Xingyou LUO, Beijing (CN); Shengji YANG, Beijing (CN); Lei WANG, Beijing (CN); Yingming LIU, Beijing (CN); Haisheng WANG, Beijing (CN); Changfeng LI, Beijing (CN)

(73) Assignees: BOE TECHNOLOGY GROUP CO., LTD., Beijing (CN); BEIJING BOE OPTOELECTRONICS TECHNOLOGY CO., LTD., Beijing (CN)

(21) Appl. No.: 15/302,997

(22) PCT Filed: Nov. 6, 2015
(86) PCT No.: PCT/CN2015/093940

§ 371 Date: Oct. 9, 2016

(30) Foreign Application Priority Data
Jun. 30, 2015 (CN) 201510374417.9

(51) Int. Cl. A61B 5/22; A61H 15/00

(52) U.S. Cl. CPC A61B 5/22; A61H 15/00; A61H 2201/5071; A61H 2015/0042

(57) ABSTRACT

A muscle detector, a massage apparatus and a method for detecting a muscle are provided. The muscle detector includes a detection component attached to a part to be detected and used to detect a muscle hardness change at the part to be detected and obtain a signal indicating a value of the muscle hardness change at the part to be detected, and a data processing component used to calculate the value of the muscle hardness change at the part to be detected according to the signal indicating the value of the muscle hardness change at the part to be detected, compare the value of the muscle hardness change at the part to be detected with a preset value, and issue a detection to-be-processed instruction in response to a determination that the value of the muscle hardness change at the part to be detected is greater than the preset value.

### CROSS-REFERENCE TO RELATED APPLICATIONS

The present application claims a priority of the Chinese patent application No. 201510374417.9 filed on Jun. 30, 2015, which is incorporated herein by reference in its entirety.

### TECHNICAL FIELD

The present disclosure relates to the field of health care products, in particular to a muscle detector, a massage apparatus and a method for detecting a muscle.

### BACKGROUND

Bodybuilding has become a fashion so far. Men get good figures, e.g., perfectly shaped abdominal and chest muscles, by means of bodybuilding. Women pursue better figures by means of bodybuilding, for example, they may lose fat effectively through running, rope skipping, dancing and the like.

A great amount of lactic acid accumulated during a period of time of strenuous exercise may result in fatigue and cause hardening of muscular tissue, and finally, muscles are formed in the body after a large amount of exercise for a long time. However, a lady that takes exercise does not expect to gain muscles at certain parts of her body. For instance, too many muscles in leg seriously affect body appearance. Therefore, how to ensure moderate exercise without causing muscle soreness and hardening and growth of muscles has been the focus of attention of numerous body builders.

### SUMMARY

Technical solutions of the present disclosure provide a muscle detector, a massage apparatus and a method for detecting a muscle, which help in monitoring moderate exercises of a sporter so as to avoid soreness and muscle rigidity caused by excessive exercise.

The present disclosure provides a muscle detector, including

a detection component, that is attached to a part to be detected and is used for detecting a muscle hardness change at the part to be detected and obtaining a signal indicating a value of the muscle hardness change at the part to be detected, and

a data processing component, that is used for calculating the value of the muscle hardness change at the part to be detected according to the signal indicating the value of the muscle hardness change at the part to be detected, comparing the value of the muscle hardness change at the part to be detected with a preset value, and issuing a detection to-be-processed instruction in response to a determination that the value of the muscle hardness change at the part to be detected is greater than the preset value.

Optionally, in the muscle detector, the detection component includes at least one pressure-sensitive sensor, the at least one pressure-sensitive sensor is attached onto the part to be detected and is used for generating an analog signal indicating the value of the muscle hardness change at the part to be detected in the case that a muscle hardness of the part to be detected is changed.

Optionally, in the muscle detector, multiple pressure-sensitive sensors are provided, and the multiple pressure-sensitive sensors are attached to multiple parts to be detected with a predetermined pressure, the multiple pressure-sensitive sensors have a one-to-one correspondence with the multiple parts to be detected, and each of the multiple pressure-sensitive sensors is used for generating the analog signal indicating the value of the muscle hardness change at the corresponding part to be detected in the case that the muscle hardness of the corresponding part to be detected is changed.

Optionally, in the muscle detector, the data processing component specifically includes:

a signal receiver, used to receive signals obtained by the multiple pressure-sensitive sensors and indicating values of the muscle hardness changes at the corresponding parts to be detected, and determine the values of the muscle hardness changes at the corresponding parts to be detected according to the signals obtained by the multiple pressure-sensitive sensors and indicating the values of the muscle hardness changes at the corresponding parts to be detected;

a data processor, used to compare the values of the muscle hardness changes at the corresponding parts to be detected with the preset value and determine a position of the pressure-sensitive sensor corresponding to at least one part to be detected in response to a determination that the value of the muscle hardness change at the at least one part to be detected in the multiple parts to be detected is greater than the preset value; and

an instruction transmitter, used to transmit the detection to-be-processed instruction, where the detection to-be-processed instruction comprises information about the position of the pressure-sensitive sensor corresponding to the at least one part to be detected.

Optionally, in the muscle detector, the detection component further includes an analog-digital converter, that is connected to the at least one pressure-sensitive sensor and is used to convert the analog signal generated by the at least one pressure-sensitive sensor for indicating the value of the muscle hardness change at the part to be detected into a digital signal and transmit the digital signal indicating the value of the muscle hardness change at the part to be detected to the data processing component.

Optionally, in the muscle detector, in the case that the multiple pressure-sensitive sensors are provided, the detection component further includes multiple of analog-digital converters, that are respectively provided between the multiple pressure-sensitive sensors and the data processing component and are used to obtain the analog signals indicating the values of the muscle hardness changes at corresponding parts to be detected from the corresponding pressure-sensitive sensors, convert the analog signals into digital signals and transmit the digital signals indicating the values of the muscle hardness changes at the corresponding parts to be detected to the data processing component.

Optionally, the muscle detector further includes a binding band in shape of a strip, where the binding band is arranged surrounding the part to be detected, and the at least one pressure-sensitive sensor is fixed on the binding band.

Optionally, the muscle detector further includes a prompt device used to receive the detection to-be-processed instruction and send warning information in response to the detection to-be-processed instruction.

Optionally, when multiple pressure-sensitive sensors are provided, the muscle detector further includes multiple prompt devices, where the quantity of the multiple prompt devices is identical to the quantity of the multiple pressure-sensitive sensors and the multiple prompt devices have a one-to-one correspondence with the multiple pressure-sensitive sensors. When the data processing component determines that the value of the muscle hardness change at at least one part to be detected in the multiple parts to be detected is greater than the preset value, the data processing component issues the detection to-be-processed instruction to the prompt device corresponding to the pressure-sensitive sensor corresponding to the at least one part to be detected, and the prompt device receiving the detection to-be-processed instruction sends warning information.

Optionally, in the muscle detector, the prompt device is a voice player or a light indicator.

The present disclosure further provides a massage apparatus including a massage device and any one of the muscle detectors mentioned above, and the massage device operates in response to the detection to-be-processed instruction of the muscle detector.

Optionally, the muscle detector also includes a controller, used to receive the detection to-be-processed instruction and initiate the massage device in response to the detection to-be-processed instruction.

Optionally, in the muscle detector, the massage device is detachably connected to the muscle detector.

The present disclosure further provides a method for detecting a muscle, comprising

obtaining a signal indicating a value of a muscle hardness change at a part to be detected through a detection of a detection component, where the detection component is attached onto the part to be detected,

calculating the value of the muscle hardness change at the part to be detected according to the signal indicating the value of the muscle hardness change at the part to be detected, and

comparing the value of the muscle hardness change at the part to be detected with a preset value, and issuing a detection to-be-processed instruction in response to a determination that the value of the muscle hardness change at the part to be detected is greater than the preset value.

Optionally, in the method for detecting the muscle, the step of issuing the detection to-be-processed instruction includes

issuing the detection to-be-processed instruction to a prompt device and sending, by the prompt device, warning information in response to the detection to-be-processed instruction, or

issuing the detection to-be-processed instruction to a massage device and starting a massage by the massage device in response to the detection to-be-processed instruction.

In the muscle detector of the present disclosure, the detection component is provided to detect the muscle hardness change (at the part to be detected). It can be determined whether the amount of exercise of the sporter is moderate according to the muscle hardness change, so that the sporter can be reminded in time and can avoid from discomfort due to soreness and muscle rigidity caused by excessive lactic acid.

### DESCRIPTION OF THE DRAWINGS

FIG. 1 is a schematic view showing a structure of a muscle detector according to some embodiments of the present disclosure;

FIG. 2 is a schematic view showing a working principle of a pressure-sensitive sensor provided in a muscle detector according to some embodiments of the present disclosure;

FIG. 3 is another schematic view showing a structure of a muscle detector according to some embodiments of the present disclosure;

FIG. 4 is a schematic view showing a structure of a massage apparatus during utilization according to some embodiments of the present disclosure;

FIG. 5 is a schematic sectional view of a massage apparatus according to some embodiments of the present disclosure; and

FIG. 6 is a schematic view showing a structure of circuits of a massage apparatus according to some embodiments of the present disclosure.

### DETAILED DESCRIPTION

In order to make technical solutions and advantages of the present disclosure more apparent, the present disclosure will be described hereinafter in conjunction with drawings and embodiments.

A muscle detector is provided according to an embodiment of the present disclosure, which includes:

a detection component, that is attached to a part to be detected and is used for detecting a muscle hardness change at the part to be detected and obtaining a signal indicating a value of the muscle hardness change at the part to be detected, and

a data processing component, that is used for calculating the value of the muscle hardness change at the part to be detected according to the signal indicating the value of the muscle hardness change at the part to be detected, comparing the value of the muscle hardness change at the part to be detected with a preset value, and issuing a detection to-be-processed instruction in response to a determination that the value of the muscle hardness change at the part to be detected is greater than the preset value.

As for the muscle detector formed by the above components, it may be determined whether a sporter moderately exercises or not by detecting the muscle hardness change at the part to be detected by the detection component, such that the sporter can be reminded in time and can avoid from discomfort due to soreness and muscle rigidity caused by excessive lactic acid.

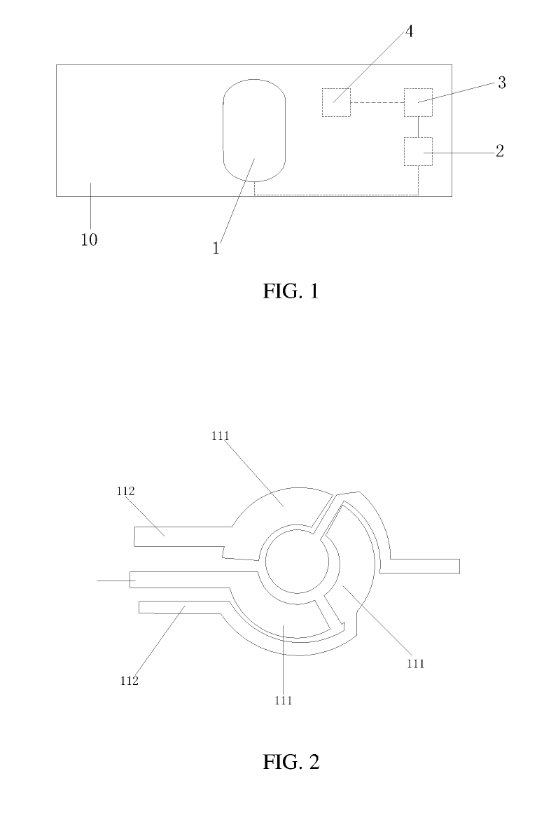

In addition, it can be understood that, the discomfort due to soreness is caused by an excessive accumulation of the lactic acid inside the muscle. The preset value, that is compared with the value of the muscle hardness change at the part to be detected to determine whether exercise of the sporter is moderate, is a critical value of muscle hardness change when the lactic acid inside the muscle reaches a preset number and the sporter starts to feel discomfort. Specifically, the preset value can be determined by means of several tests. FIG. 1 is a schematic view showing a structure of a muscle detector according to an embodiment of the present disclosure. In the muscle detector according to the embodiment of the present disclosure, the detection component includes at least one pressure-sensitive sensor 1. Specifically, the muscle detector further includes a binding band 10 in shape of a strip, and the pressure-sensitive sensor 1 is fixedly provided on the binding band 10. For easily arranging the binding band 10 surrounding the part to be detected, the binding band 10 generally is made of a soft cloth, and the pressure-sensitive sensor 1 can be fixed on the binding band 10 in a manner of sewing or sticking.

When using the above structured muscle detector, the part to be detected, for example, a certain part of the body (a leg or an arm) is coated and surrounded by the binding band 10 and the pressure-sensitive sensor 1 is located at the position of the muscle of the body, where two ends of the binding band 10 are fixed to each other and the whole binding band 10 is fixed on the body. Specifically, the two ends of the binding band 10 may be fixed to each other by tying or through adhesive connection between adhesive buttons respectively provided at the two ends of the binding band 10. The fixation of the binding band 10 also can be realized in other manners that are not detailed herein.

In addition, when the binding band 10 is attached to the body, optionally, the pressure-sensitive sensor 1 is attached at a muscle position (the part to be detected) of the body with a predetermined pressure, so that a detection of the pressure-sensitive sensor 1 is relatively sensitive.

FIG. 2 is a schematic structural view of the pressure-sensitive sensor. As shown in FIG. 2, the pressure-sensitive sensor generally includes at least two sensitive components 111 in parallel. The sensitive components 111 are capacitive pressure-sensitive components or resistive pressure-sensitive components. The sensitive components 111 are used to sense a hardness change of the muscle at the part to be detected, and the value of the muscle hardness change is converted into a voltage signal. An analog signal indicating the value of the muscle hardness change is generated and output from a lead 112 of the pressure-sensitive sensor.

A person skilled in the art is familiar with the working principle of the pressure-sensitive sensor. Thus, relevant description is not repeated here.

As for the muscle detector according to the embodiment of the present disclosure, the pressure-sensitive sensor is attached onto the part to be detected, and the value of the muscle hardness change is converted, using the property of the pressure-sensitive sensor, into the voltage signal that can be output and read; hence, the amount of exercise can be indirectly reflected by the muscle hardness change.

Besides, in the muscle detector of the present disclosure, the detection component provided for detecting the muscle hardness change at the part to be detected is not merely limited to the pressure-sensitive sensor. It also can be a common pressure sensor.

As shown in FIG. 1, besides the pressure-sensitive sensor 1, the detection component of the muscle detector according to the embodiment of the present disclosure also includes an analog-digital converter 2 electrically connected to the pressure-sensitive sensor 1. The analog-digital converter 2 is connected to the lead 112 (as shown in FIG. 2) of the pressure-sensitive sensor 1 and is for obtaining the analog signal indicating the value of the muscle hardness change at the part to be detected from the pressure-sensitive sensor 1, converting the analog signal into a digital signal and transmitting the digital signal indicating the value of the muscle hardness change at the part to be detected to a data processing component 3.

The data processing component 3 is used for calculating the value of the muscle hardness change at the part to be detected according to the digital signal indicating the value of the muscle hardness change at the part to be detected, comparing the calculated value of the muscle hardness change at the part to be detected with the preset value, and issuing the detection to-be-processed instruction in response to a determination that the value of the muscle hardness change at the part to be detected is greater than the preset value. The preset value is a critical value of a muscle hardness change when the lactic acid inside the muscle is accumulated to a preset amount and the sporter starts to feel discomfort. Specifically, the preset value can be determined through several tests.

As shown in FIG. 1, the data processing component 3 and the analog-digital converter 2 are both fixed inside the binding band 10. A person skilled in the art should be familiar with the manner of fixing the two components inside the binding band 10 and the manner of electrically connecting the two components to the pressure-sensitive sensor 1. Relevant description is not repeated here.

Optionally, in an embodiment of the present disclosure, as shown in FIG. 1, the muscle detector further includes a prompt device 4, which is fixed with respect to the binding band 10 and electrically connected to the data processing component 3. The prompt device 4 receives the detection to-be-processed instruction issued by the data processing component 3 and sending warning information according to the detection to-be-processed instruction to remind the sporter that the lactic acid in the part to be detected at this time has reached a preset amount and that the exercise has reached a critical amount with which the discomfort of the muscle will be caused and the muscle will be hardened. For avoiding the discomfort, the prompt device 4 prompts the sporter to stop and take a rest.

Specifically, the prompt device 4 may be a voice player or a light prompter, and it can give a prompt to the sporter by playing a voice or light flickering.

With the muscle detector in the above embodiments, the sporter can fix the binding band provided with the pressure-sensitive sensor on his body before taking exercise so that the pressure-sensitive sensor can be pressed on the muscle position of the body with a predetermined pressure. During the exercise, long time and large amount of exercise results in increased lactic acid in the muscle. When the pressure-sensitive sensor detects that the value of the muscle hardness change at the part to be detected reaches the preset value, the prompt device may send a warning prompt.

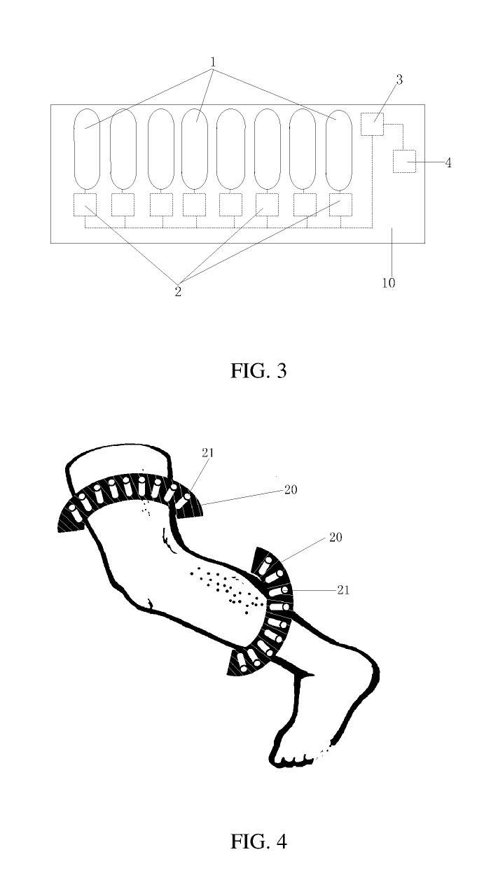

Furthermore, as shown in FIG. 3, the present disclosure provides in some embodiments a muscle detector. In the embodiments, the detection component includes multiple pressure-sensitive sensors 1 that are distributed and fixed at different positions of the binding band 10. The structure of the binding band 10 is the same as that in the previous embodiments.

When two ends of the binding band 10 are fixed to each other and the whole binding band 10 is fixed on the body, the multiple pressure-sensitive sensors 1 are attached onto multiples parts to be detected with a predetermined pressure, and the multiple pressure-sensitive sensors 1 have one-to-one correspondence with the multiple parts to be detected.

The multiple pressure-sensitive sensors 1 are electrically connected to the data processing component 3 respectively. The working principle of each of the multiple pressure-sensitive sensors 1 is the same as that in the previous embodiments, and it is not repeated here. Each of the multiple pressure-sensitive sensors 1 is used for detecting and obtaining a signal indicating the value of the muscle hardness change at a corresponding part to be detected and transmitting the signal indicating the value of the muscle hardness change at the corresponding part to be detected to the data processing component 3.

Specifically, the data processing component 3 includes:

a signal receiver, for receiving signals obtained by the multiple pressure-sensitive sensors 1 and indicating values of the muscle hardness changes at the corresponding parts to be detected, and determining the values of the muscle hardness changes at the corresponding parts to be detected according to the signals obtained by the multiple pressure-sensitive sensors 1 and indicating the values of the muscle hardness changes at the corresponding parts to be detected,

a data processor, for comparing the values of the muscle hardness changes at the corresponding parts to be detected with a preset value and determining a position of the pressure-sensitive sensor 1 corresponding to at least one part to be detected in response to a determination that the value of the muscle hardness change at the at least one part to be detected in the multiple parts to be detected is greater than the preset value, and

an instruction transmitter for transmitting the detection to-be-processed instruction, where the detection to-be-processed instruction includes information about the position of the pressure-sensitive sensor 1 corresponding to the at least one part to be detected.

With the muscle detector according to the above embodiment, multiple pressure-sensitive sensors 1 are provided and the multiple pressure-sensitive sensors 1 are distributed and fixed at different parts of the body when the binding band 10 is fixed around the body. The multiple pressure-sensitive sensors 1 detect values of muscle hardness changes at different parts to be detected. For example, the binding band 10 can be fixed around the waist, and the pressure-sensitive sensors 1 distributed at different positions of the waist can detect the values of the muscle hardness changes at the different positions of the waist. Besides, the muscle of one part can be provided with multiple pressure-sensitive sensors arranged in a distributed manner, and then, the muscle hardness change during exercise can be detected and determined more accurately.

In addition, as shown in FIG. 3, the detection component of the muscle detector according to the embodiments further includes multiple analog-digital converters 2, which are respectively provided between the multiple pressure-sensitive sensors 1 and the data processing component 3 for obtaining analog signals indicating values of the muscle hardness changes at corresponding parts to be detected from the corresponding pressure-sensitive sensors 1, converting the analog signals into digital signals and transmitting the digital signals indicating the values of the muscle hardness changes at the corresponding parts to be detected to the data processing component 3.

Besides, as shown in FIG. 3, the muscle detector in the embodiments further includes:

a prompt device 4, which is electrically connected to the data processing component 3 and is for receiving the detection to-be-processed instruction issued by the data processing component 3 and sending warning information according to the detection to-be-processed instruction.

Specifically, in some embodiments, there may be only one prompt device 4. When any one of the multiple pressure-sensitive sensors 1 detects that the value of the muscle hardness change at the corresponding part to be detected is greater than the preset value, the data processing component 3 issues the detection to-be-processed instruction according to a detection result of the pressure-sensitive sensor 1 so that the prompt device 4 sends the warning information. Optionally, there may be multiple prompt devices 4. The quantity of the prompt devices 4 is identical to the quantity of the pressure-sensitive sensors 1, the multiple prompt devices 4 have a one-to-one correspondence with the multiple pressure-sensitive sensors 1, and positions of the prompt devices 4 correspond to positions of the pressure-sensitive sensors 1. When one pressure-sensitive sensor 1 detects that the value of the muscle hardness change at the corresponding part is greater than the preset value, the data processing component 3 issues the detection to-be-processed instruction to the prompt device 4 corresponding to the one pressure-sensitive sensor 1 according to the detection result of the one pressure-sensitive sensor 1, and only the prompt device 4 corresponding to the one pressure-sensitive sensor 1 is enabled to send the warning information.

With the muscle detector according to the embodiments, the sporter can detect muscle hardness changes at different parts using one muscle detector, or multiple pressure-sensitive sensors can be provided in a distributed manner at one part so as to accurately detect and determine the muscle hardness change at the one part.

In another aspect, the present disclosure further provides in some embodiments a massage apparatus, which includes a massage device and further includes the muscle detector in the above embodiments.

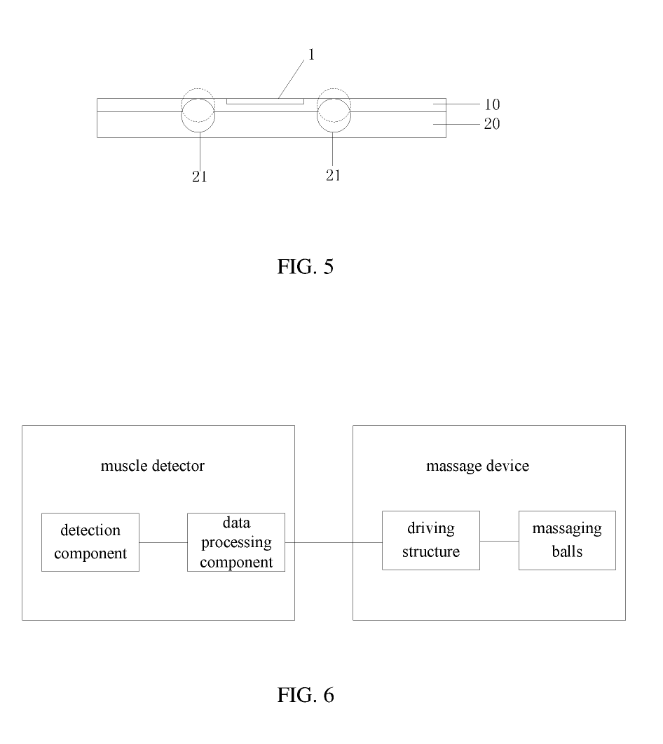

FIG. 4 is a schematic view showing a structure of a massage apparatus provided in the present disclosure, which is sleeved on a leg, and FIG. 5 is a schematic sectional view of the massage apparatus. As shown in FIG. 4 and FIG. 5, the massage apparatus includes a massage device. The massage device includes a base 20 and movable massaging balls 21 provided on the base 20. The massaging balls 21 are driven by a driving structure and are for massaging or stretching the muscle. In addition, the massage apparatus further includes a muscle detector that is connected to the massage device and has the structure described in the above embodiments. A binding band 10 of the muscle detector is attached onto the base 20 of the massage device.

The specific structure of the muscle detector is given in the foregoing embodiments and is not repeated here. In the massage apparatus according to the embodiments of the present disclosure, as shown in FIG. 6, the detection component of the muscle detector is electrically connected to the data processing component, and the data processing component of the muscle detector is electrically connected to the driving structure driving the massaging balls 21, where the driving structure is used for receiving the detection to-be-processed instruction issued by the data processing component and initiating the massaging balls 21 to carry out a massaging or stretching operation.

Specifically, the massage apparatus having the muscle detector provided in the present disclosure can be sleeved on the body (at the position of the leg shown in FIG. 4) as shown in FIG. 4. When the sporter takes exercise, the massage apparatus is sleeved on his leg and a pressure-sensitive sensor 1 provided on the binding band 10 of the muscle detector directly contacts the body. The pressure-sensitive sensor 1 is used for detecting the muscle hardness change of the leg muscle, obtaining the value of the muscle hardness change and determining the amount of exercise of the sporter. When the data processing component detects that the value of the muscle hardness change is greater than the preset value, the detection to-be-processed instruction is transmitted to the driving structure of the massage device. The driving structure drives the massaging balls 21 to massage or stretch the muscle in response to the detection to-be-processed instruction.

Optionally, the driving structure also can drive each of the massaging balls 21 to act between two positions, such that the arrangement of the massaging balls 21 may not influence the attachment effect between the pressure-sensitive sensor 1 and the part to be detected. As shown in FIG. 5, when the pressure-sensitive sensor 1 detects the muscle hardness, the driving structure enables the massaging balls 21 to be located at positions shown by solid circles in FIG. 5, i.e. they are provided below an upper surface of the pressure-sensitive sensor 1. When there is the need to enable the massaging balls 21 to stretch or massage the muscle, the massaging balls 21 can be located at positions shown by dotted circles in FIG. 5, i.e. they protrude out of the upper surface of the pressure-sensitive sensor 1, so that the massaging balls 21 can perform massaging or stretching operation.

Further optionally, the muscle detector and the massage device according to the above embodiments may be connected to each other through a detachable structure. Before taking exercise, the muscle detector can be removed from the massage apparatus and thus separated from the massage device, and then may be fixed on the human body for detecting the muscle hardness changes during the exercise. After the exercise, when the prompt device of the muscle detector prompts that the muscle hardness change after the exercise exceeds the preset value, the massage device can be fixed on the body for massaging.

Further optionally, when the muscle detector of the massage apparatus is provided with multiple pressure-sensitive sensors 1, the driving structure for the massaging ball 21 can drive a part of the massaging balls 21 to perform the massaging and stretching operations according to the detection result of the muscle detector, so that only the muscles where the muscle hardness changes exceed the preset value are massaged or stretched and the lactate metabolism is accelerated.

With the massage apparatus according to the embodiments of the present disclosure, the exercised part of the human body can be massaged or stretched according to the detection result of the muscle detector, so that the exercised part can be kept good shape and discomfort after the exercise can be eliminated.

The present disclosure further provides a method for detecting a muscle, including:

obtaining a signal indicating a value of a muscle hardness change at a part to be detected through a detection of a detection component, where the detection component is attached onto the part to be detected,

calculating the value of the muscle hardness change at the part to be detected according to the signal indicating the value of the muscle hardness change at the part to be detected, and

comparing the value of the muscle hardness change at the part to be detected with a preset value, and issuing a detection to-be-processed instruction in response to a determination that the value of the muscle hardness change at the part to be detected is greater than the preset value.

Specifically, the step of issuing a detection to-be-processed instruction includes:

issuing the detection to-be-processed instruction to a prompt device, so that the prompt device sends warning information in response to the detection to-be-processed instruction, or

issuing the detection to-be-processed instruction to a massage device, so that the massage device starts massaging in response to the detection to-be-processed instruction.

With the method for detecting muscles, it can be determined whether the amount of exercise of the sporter is moderate, so that the sporter can be reminded in time and can avoid from discomfort due to soreness and muscle rigidity caused by excessive lactic acid.

The above are merely preferred embodiments of the present disclosure. It should be noted that, a person skilled in the art may make various modifications of and improvements to the present disclosure without departing from the principle of the present disclosure, and all these modifications and improvements fall in the protection scope of the present disclosure.

1. A muscle detector, comprising:
a detection component, that is attached to a part to be detected and is configured to detect a muscle hardness change at the part to be detected and obtain a signal indicating a value of the muscle hardness change at the part to be detected; and
a data processing component, that is configured to calculate the value of the muscle hardness change at the part to be detected according to the signal indicating the value of the muscle hardness change at the part to be detected, compare the value of the muscle hardness change at the part to be detected with a preset value, and issue a detection to-be-processed instruction in response to a determination that the value of the muscle hardness change at the part to be detected is greater than the preset value.

2. The muscle detector according to claim 1, wherein the detection component comprises at least one pressure-sensitive sensor, the at least one pressure-sensitive sensor is attached onto the part to be detected and is configured to generate an analog signal indicating the value of the muscle hardness change at the part to be detected in the case that a muscle hardness of the part to be detected is changed.

3. The muscle detector according to claim 2, wherein the number of the at least one pressure-sensitive sensor is plural, and the plurality of pressure-sensitive sensors is attached to a plurality of parts to be detected with a predetermined pressure, the plurality of pressure-sensitive sensors has a one-to-one correspondence with the plurality of parts to be detected, and each of the plurality of pressure-sensitive sensors is configured to generate the analog signal indicating the value of the muscle hardness change at the corresponding part to be detected in the case that the muscle hardness of the corresponding part to be detected is changed.

4. The muscle detector according to claim 3, wherein the data processing component comprises:
a signal receiver, configured to receive signals obtained by the plurality of pressure-sensitive sensors and indicating values of the muscle hardness changes at the corresponding parts to be detected, and determine the values of the muscle hardness changes at the corresponding parts to be detected according to the signals obtained by the plurality of pressure-sensitive sensors and indicating the values of the muscle hardness changes at the corresponding parts to be detected;
a data processor, configured to compare the values of the muscle hardness changes at the corresponding parts to be detected with the preset value and determine a position of the pressure-sensitive sensor corresponding to at least one part to be detected in response to a determination that the value of the muscle hardness change at the at least one part to be detected in the plurality of parts to be detected is greater than the preset value; and
an instruction transmitter, configured to transmit the detection to-be-processed instruction, wherein the detection to-be-processed instruction comprises information about the position of the pressure-sensitive sensor corresponding to the at least one part to be detected.

5. The muscle detector according to claim 2, wherein the detection component further comprises an analog-digital converter, that is connected to the at least one pressure-sensitive sensor and is configured to convert the analog signal generated by the at least one pressure-sensitive sensor for indicating the value of the muscle hardness change at the part to be detected into a digital signal and transmit the digital signal indicating the value of the muscle hardness change at the part to be detected to the data processing component.

6. The muscle detector according to claim 3, wherein the detection component further comprises a plurality of analog-digital converters, that is respectively provided between the plurality of pressure-sensitive sensors and the data processing component and is configured to obtain the analog signals indicating the values of the muscle hardness changes at corresponding parts to be detected from the corresponding pressure-sensitive sensors, convert the analog signals into digital signals and transmit the digital signals indicating the values of the muscle hardness changes at the corresponding parts to be detected to the data processing component.

7. The muscle detector according to claim 2, further comprising a binding band in shape of a strip, wherein the binding band is arranged surrounding the part to be detected, and the at least one pressure-sensitive sensor is fixed on the binding band.

8. The muscle detector according to claim 1, further comprising a prompt device configured to receive the detection to-be-processed instruction and send warning information in response to the detection to-be-processed instruction.

9. The muscle detector according to claim 3, further comprising a plurality of prompt devices, wherein the quantity of the plurality of prompt devices is identical to the quantity of the plurality of pressure-sensitive sensors and the plurality of prompt devices has a one-to-one correspondence with the plurality of pressure-sensitive sensors;
wherein when the data processing component determines that the value of the muscle hardness change at at least one part to be detected in the plurality of parts to be detected is greater than the preset value, the data processing component issues the detection to-be-processed instruction to the prompt device corresponding to the pressure-sensitive sensor corresponding to the at least one part to be detected, and the prompt device receiving the detection to-be-processed instruction sends warning information.

10. The muscle detector according to claim 8, wherein the prompt device is a voice player or a light indicator.

11. A massage apparatus, comprising a massage device and a muscle detector, wherein the muscle detector comprises:
a detection component, that is attached to a part to be detected and is configured to detect a muscle hardness change at the part to be detected and obtain a signal indicating a value of the muscle hardness change at the part to be detected; and
a data processing component, that is configured to calculate the value of the muscle hardness change at the part to be detected according to the signal indicating the value of the muscle hardness change at the part to be detected, compare the value of the muscle hardness change at the part to be detected with a preset value, and issue a detection to-be-processed instruction in response to a determination that the value of the muscle hardness change at the part to be detected is greater than the preset value; and
wherein the massage device operates in response to the detection to-be-processed instruction of the muscle detector.

12. The massage apparatus according to claim 11, further comprising a controller configured to receive the detection to-be-processed instruction and initiate the massage device in response to the detection to-be-processed instruction.

13. The massage apparatus according to claim 11, wherein the massage device is detachably connected to the muscle detector.

14. A method for detecting a muscle, comprising:
obtaining a signal indicating a value of a muscle hardness change at a part to be detected through a detection of a detection component, wherein the detection component is attached onto the part to be detected;
calculating the value of the muscle hardness change at the part to be detected according to the signal indicating the value of the muscle hardness change at the part to be detected; and
comparing the value of the muscle hardness change at the part to be detected with a preset value, and issuing a detection to-be-processed instruction in response to a determination that the value of the muscle hardness change at the part to be detected is greater than the preset value.

15. The method for detecting the muscle according to claim 14, wherein the step of issuing the detection to-be-processed instruction comprises:
issuing the detection to-be-processed instruction to a prompt device and sending, by the prompt device, warning information in response to the detection to-be-processed instruction, or
issuing the detection to-be-processed instruction to a massage device and starting a massage by the massage device in response to the detection to-be-processed instruction.

16. The muscle detector according to claim 9, wherein the prompt device is a voice player or a light indicator.
